(19) United States
(12) Patent Application Publication
(10) Pub. No.: US 2011/0230806 A1
(43) Pub. Date: Sep. 22, 2011

(54) Apparatus and Method for Monitoring and Active Correction of Orthosis to Body Pressure Inside of An Orthosis

(76) Inventors: Edmond Hok Ming Lou, Edmonton (CA); Mark Vernon Fedorak, Edmonton (CA); Nicholas Chi-Kin Lo, Edmonton (CA); Christopher Charles Kirchen, Sherwood Park (CA); Garth Eldor Thomas, St. Albert (CA)

(21) Appl. No.: 12/729,063

(22) Filed: Mar. 22, 2010

(51) Int. Cl. A61F 5/01

(52) U.S. Cl. 602/13

(57) ABSTRACT

A system and method is provided for monitoring and controlling the pressure inside of an orthosis or orthotic brace. The system can measure the pressure inside of the brace using a force sensor and then, using an air bladder placed between the outside wall of the orthotic brace and the body of the person wearing the brace, deflate or inflate the air bladder as required to maintain a constant pressure between the brace and the body.

### TECHNICAL FIELD

The present disclosure is directed to the field of orthosis used in the treatment of physiological disorders, in particular, orthosis used in the treatment of idiopathic scoliosis.

### BACKGROUND

Idiopathic scoliosis (“IS”) is a three-dimensional curvature of the spine with vertebral rotation for which there is no known cause. This curvature affects the rib cage and presents deformities of the trunk. Treatment modalities for scoliosis are based on patient's physiologic maturity, curve severity, curve location, surface deformities, and the risk of progression. Orthotic treatment (“orthosis”) is the most commonly used non-surgical method to control curve progression during the high-risk growing phase of patients. An orthosis or brace is a hard polypropylene plastic shell. Localized pressure pads are usually added inside the orthosis to provide mechanical support to the spine. An orthosis's corrective action is thought to have both “passive” and “active” components. The “passive” component is the orthosis's ability to provide mechanical support to the body; the “active” component involves the patient's own adjustment of his/her body position in reaction to wearing the orthosis (i.e pulling away from the orthosis's pressure sites). A properly fitted orthosis should reduce the curve with the goal of orthosis treatment being generally accepted to prevent further curve progression. To be effective, the orthosis must be worn as prescribed and until the child has completed growth. However, some studies [1], [2] have questioned its effectiveness, whereas others [3], [4], [5] have concluded that the use of an orthosis effectively attenuates the natural progression of AIS. The efficacy of orthotic treatment remains a controversial topic among pediatric orthopaedic surgeons.

The most important determinant of effectiveness of orthotic management is compliance, both in terms of “quantity” (ie. wearing the orthosis for the prescribed amount of time per day for the full duration of the treatment cycle) and “quality” (ie. while being worn the orthosis is providing the intended curve correction). To assess compliance, the most commonly accepted methods are to ask the patient or family if the orthosis is being used and to visually inspect for signs of wear. To investigate how tightly patients wear their orthosis during their normal usage, patients at the scoliosis clinics are asked to demonstrate how they tighten their orthoses. A mark is usually placed on the straps to help patients to properly tighten their orthoses. However, stretching of the orthosis straps or thinning of the lining foam may occur after the orthosis has been used for a few months, causing the mark to be inaccurate.

Other methods [6], [7], [10], [11], [12] have been used to study either loads or compliance while patients wore their orthoses, however, these methods had limitations (eg. could only be used in the laboratory environment) and none of them measured the quantity and quality of orthosis usage at the same time. To study both the quantity and quality issues, a low-powered portable load monitoring system has been developed [8]. Clinical trials indicated that patients who wore their orthoses more frequently as well as tightened to the prescribed tension either improved or stabilized their spinal curvature [9].

It is, therefore, desirable to provide a “smart computerized orthosis” that maintains constant prescribed loads on the pressure pad imposed on the trunk during daily living.

### SUMMARY

An apparatus and method is provided that assists with solving one of the key problems with modern day orthosis; that is ensuring the “quality” and “quantity” of a patients wear is sufficient to correct their orthotic condition. “Quality” of orthosis wear is defined as wearing the orthosis at the prescribed tightness, and “quantity” is defined as the amount of time the orthosis is worn. Both are important in that if the orthosis is worn extensively but at too low a tightness the orthosis will likely not meet its treatment goals. The same can be said if the orthosis is worn at the prescribed tightness but not frequently enough to be effective.

As the patient wears their orthosis throughout the day, the pressure inside the orthosis can change due to the position of the body changing throughout the day (sitting, lying down, etc.), and the holding straps on the orthosis loosing as the day goes on. The apparatus and method presented herein attempts to ensure that, while the patient is wearing the orthosis, the force inside the orthosis (the orthosis-body pressure) is sufficient as to ensure that the quality of brace wear will be high. This can be accomplished by having a force sensor located at the orthosis pressure point, and having the orthotist set a target force level and the range of acceptable force levels between the body and the orthosis, and then having the device monitor the actual force levels against the target force levels throughout the day.

If the apparatus detects that the force inside of the orthosis is too low over a period of time, then the apparatus can inflate an air bladder that is located at the pressure pad point inside of the orthosis. If the apparatus detects that the force inside of the orthosis is too high over a period of time, then the apparatus will deflate the air bladder that is located at the pressure pad point inside of the orthosis. For orthosis with multiple pressure pad points, the apparatus can support this by having multiple force sensors and multiple air bladders.

In order to inflate and deflate the air bladder, the apparatus can comprise a main controller board that contains the electronics to run a pump and holding valve and to take readings from a force or pressure sensor. The main controller device can comprise of a microcontroller, storage memory, real time clock, energy storage device and communications circuitry that can allow the apparatus to record sensor readings, analyze the readings and then perform a correction as required. The main controller board can also allow the device to communicate with an external secondary device such as but not limited to a personal computer, cellular phone or a watch. These external devices can be used to configure the apparatus such as setting the time, sampling rate and the target force levels or display data collected by the device such as, but not limited to, orthosis wear characteristics, and number of corrections made. The main body of the apparatus can contain the main controller board, pump and valves, which can be located separately from the pressure pad area that contains the force sensor and the air bladder.

To complete a correction, the microcontroller can read the force sensor and if the force sensor records a reading that is too low over a period of time, then the microcontroller can open a holding valve that maintains the pressure in the system and then turn on a pump. The apparatus can then take force readings and determine if the force is correct, if it is the force is not high enough the pump will be left on until such point that the readings become within the acceptable range. In some embodiments of the apparatus, the pump can be stopped between force readings to allow the system to stabilize. This process can be repeated until the force readings are within an acceptable range. To prevent over inflation of the apparatus, the apparatus can further comprise an air pressure bleeder valve that can limit the total pressure within the system. In addition, if the force sensor is not able to reach or change the pressure within the brace because the system is at maximum pressure, or if the brace is not on the body, then the pump and valve can turn off until such time that the patient corrects the issue.

If the force sensor records a reading that is too high over a period of time then, the microcontroller can open a holding valve that maintains the pressure in the system and allow air to bleed out of the system through the bleeder/relief valve. If the holding valve stays open for a long duration and no change in pressure is found, then the system can close the holding valve as the patient is wearing the brace at over the target force level and the air bladder is fully deflated.

In order to simplify installation of the apparatus on the orthosis, a template can be provided that allows an orthotist to drill and cut the orthosis correctly to fit the device main body and the sensor actuator assembly. In addition, to simplify routing of the air hose and sensor wire from the device main body to the sensor actuator assembly (or assemblies), a dual channel hose can be provided that allows for the air to flow through one side of the hose and the sensor wire or cable to run through the other side of the hose. Then, a correctly sized flexible rod can be supplied such that the orthotist can use to mold a channel in the shell of the orthosis in which to route the dual channel hose from the device main body to the sensor actuator assembly.

Often, an orthopaedic surgeon will request that X-Rays be taken while the patient is in the brace and, as such, the device main body must be removed whilst maintaining the air pressure in the bladder. In order to accomplish this, the apparatus can be provided with easy to remove screws on the front and an easy to remove connector that includes both the sensor signals and air in a single connector. By clipping the air-side of the dual channel hose shut, and removing the apparatus from the orthosis, the X-Ray can be taken with only a very minimal impact on X-Ray quality.

Broadly stated, an apparatus is provided for monitoring and controlling the pressure applied to a patient by an orthosis, comprising: a main body assembly configured to be operatively disposed on the orthosis, the main body assembly further comprising: an enclosure configured to be releasably attached to the orthosis, an air pump disposed within the enclosure, an air holding valve disposed within the enclosure and operatively connected to the air pump, a first connector disposed on the enclosure and operatively connected to the air holding valve, the first connector further comprising at least one electrical connector, a controller disposed within the enclosure for controlling the air pump and the air holding valve, the controller operatively connected to the at least one electrical connector, and an energy source device disposed within the enclosure and operatively connected to the controller, the air pump and the air holding valve; and a sensor/actuator assembly, comprising: at least one air bladder configured to be disposed on the orthosis, the at least one air bladder further configured to provide pressure to the patient, at least one force sensor operatively disposed on the at least one air bladder, the at least one force sensor configured to measure the pressure the at least one air bladder exerts on the patient wearing the orthosis, a hose assembly having first and second ends, the first end operatively connected to the at least one air bladder and the at least one force sensor, the hose assembly further comprising an air hose operatively connected to the at least one air bladder and a conduit operatively connected to the at least one force sensor, a second connector operatively connected to the second end of the hose assembly and configured to releasably connect with the first connector, the second connector configured to provide communication from the air holding valve to the at least one air bladder when the second connector is connected to the first connector, and at least one electrical conductor disposed within the conduit operatively connecting the at least one force sensor to the second connector whereby an electrical connection is made between the at least one force sensor and the controller when the second connector is connected to the first connector.

Broadly stated, a method is provided for monitoring and controlling the pressure applied to a patient by an orthosis disposed on the patient, the method comprising the steps of: measuring the air pressure of at least one air bladder disposed on the orthosis for applying pressure to the patient; and inflating the at least one air bladder with air until the air pressure is within the predetermined air pressure range if the air pressure is below a predetermined air pressure range.

Broadly stated, an apparatus is provided for monitoring and controlling the pressure applied to a patient by an orthosis disposed on the patient, comprising: means for measuring the air pressure of at least one air bladder disposed on the orthosis for applying pressure to the patient; and means for inflating the at least one air bladder with air until the air pressure is within the predetermined air pressure range if the air pressure is below a predetermined air pressure range.

### BRIEF DESCRIPTION OF DRAWINGS

FIG. 1A is a top perspective view depicting an apparatus for controlling air bladders in an orthosis.

FIG. 1B is a bottom perspective view depicting the apparatus of FIG. 1A.

FIG. 1C is a perspective view depicting a sensor/actuator assembly for the apparatus of FIG. 1A.

FIG. 1D is a front perspective view depicting an orthosis fitted on the torso of a patient comprising the apparatus of FIG. 1A.

FIG. 1E is a rear perspective view depicting the orthosis of FIG. 1D.

FIG. 2A is a perspective view depicting a connector assembly for the apparatus of FIG. 1A.

FIG. 2B is an exploded perspective view depicting the connector assembly of FIG. 2A.

FIG. 3A is an exploded perspective view depicting a main controller printed circuit board and wiring harness for the apparatus of FIG. 1.

FIG. 3B is an exploded perspective view depicting the main controller printed circuit board of FIG. 3A and an energy storage device.

FIG. 4 is a top perspective view depicting the internal components of the apparatus of FIG. 1A.

FIG. 5 is an exploded perspective view depicting the apparatus of FIG. 1A.

FIG. 6A is a top plan view depicting one embodiment of a bleeder relief air valve used with the apparatus of FIG. 1A, and a cross-section view thereof along section lines A-A.

FIG. 6B is a top plan view depicting a t-nut and screw assembly used with the apparatus of FIG. 1A, and a cross-section view thereof along section lines B-B.

FIG. 7A is a pneumatic schematic diagram depicting the air supply system for the apparatus of FIG. 1A when the air valve is closed.

FIG. 76 is a pneumatic schematic diagram depicting the air supply system for the apparatus of FIG. 1A when the air valve is open.

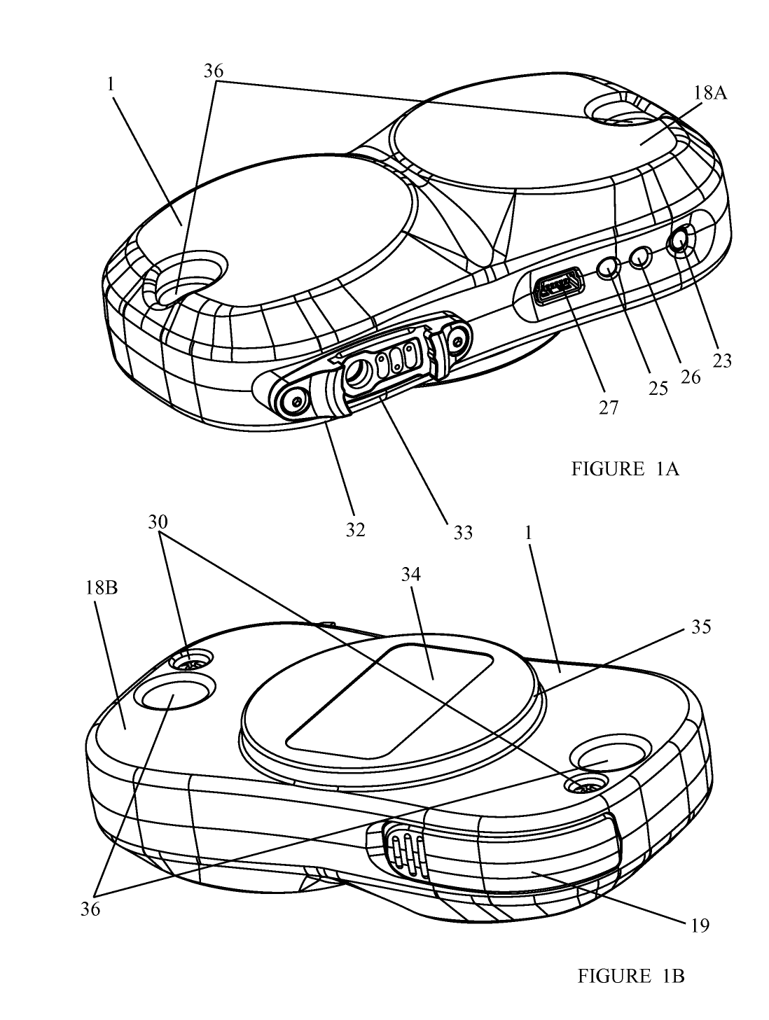

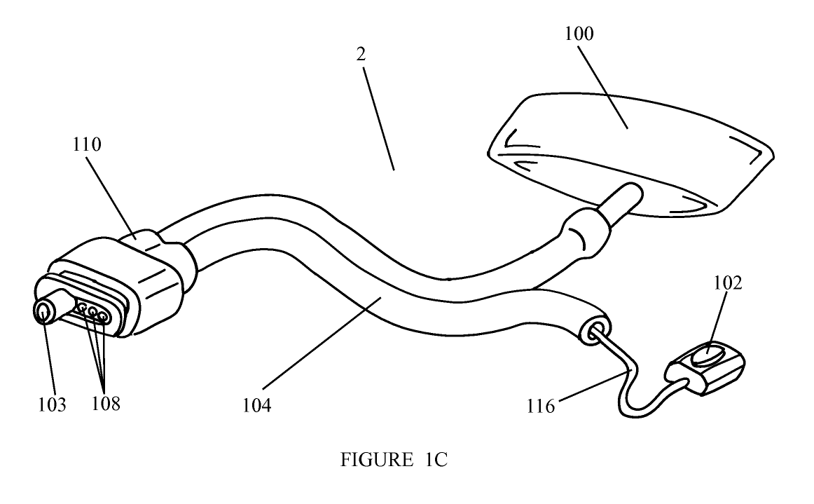

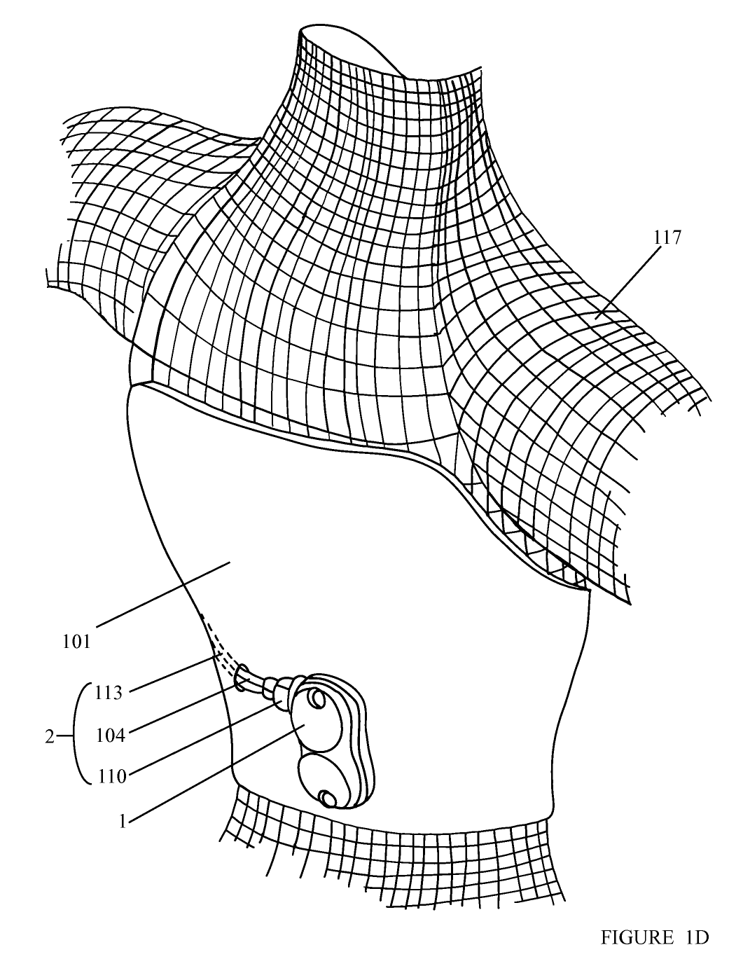

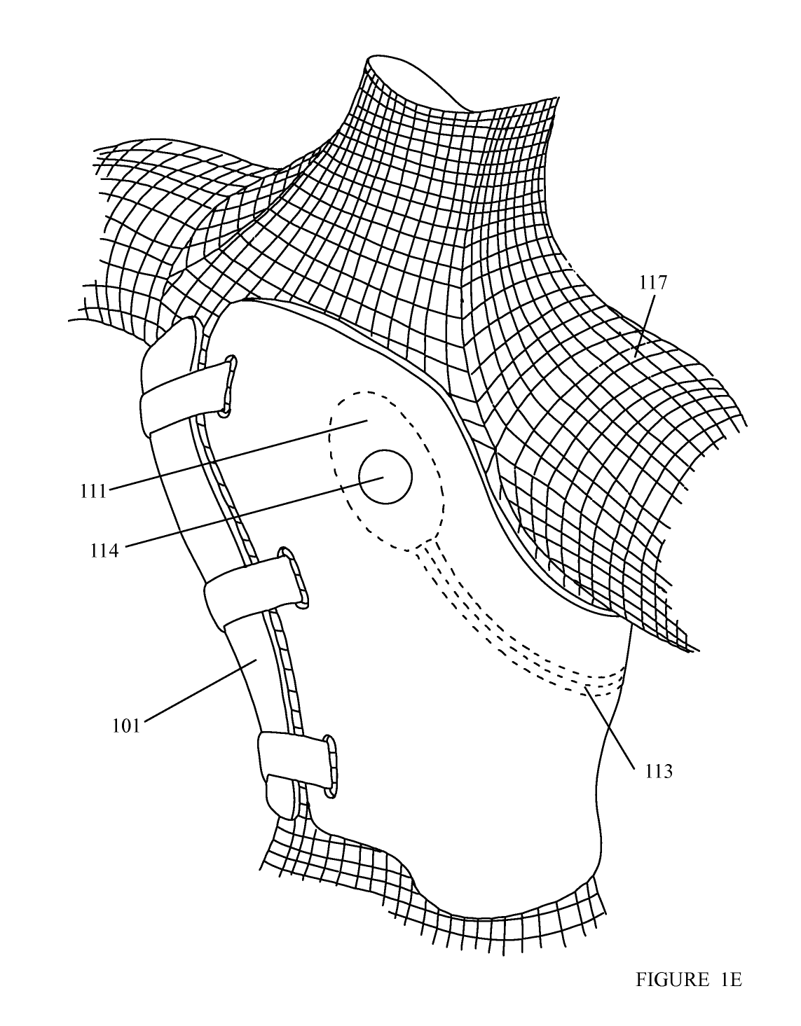

FIG. 8A is a side elevation view depicting the apparatus of FIG. 1A assembled on an orthosis.

FIG. 8B is a cross-section top plan view depicting a force sensor and an air bladder as assembled on an orthosis for use with the apparatus of FIG. 1A.

FIG. 8C is a side elevation view depicting the orthosis, force sensor and air bladder of FIG. 8B.

FIG. 9A is a flowchart depicting the operation of the apparatus of FIG. 1A while in Run Mode.

FIG. 9B is a flowchart depicting the operation of the apparatus of FIG. 1A when a button is depressed on the apparatus.

### DETAILED DESCRIPTION

An apparatus and method for monitoring and active correction of orthosis to body pressure inside of an orthosis is provided and as described herein. In one embodiment, the apparatus can comprise of two main assemblies. The first assembly can comprise device main body (1) as depicted in FIGS. 1A and 18, and the second assembly can comprise sensor/actuator assembly (2) as depicted in FIG. 10.

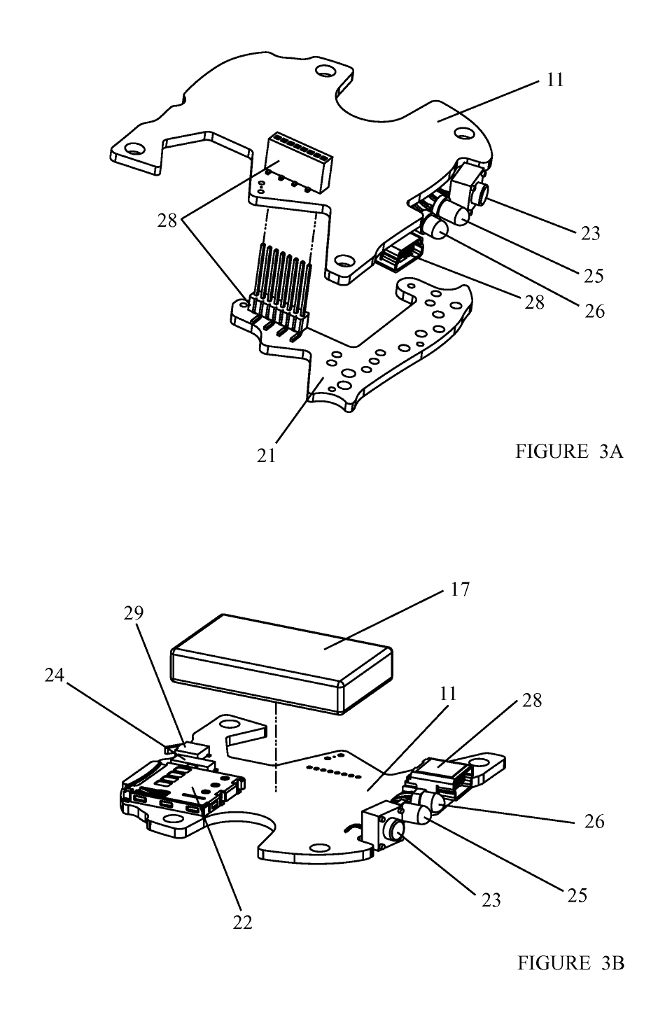

Referring to FIGS. 1A through 5, main body (1) can comprise main controller circuit board (11), one or more pumps (12), one or more holding valves (13), one or more sensor/actuator connectors (14), one or more interconnect tubes (15), one or more air bleeder/relief valves (16), energy storage device (17), enclosure (18A,18B) which can be composed of any one or combination of thermoplastic, metal or any other suitable material, flexible protective door (19), mounting hardware (20) and wiring harness (21).

Main controller circuit board (11) can comprise the controller and communication circuitry for the device. Main controller circuit board (11) can comprise a micro-controller unit (“MCU”) that can connect all of the systems together. The MCU can be any 8, 16 or 32 bit processor such as the Atmel Inc. ATmega644V or any functionally equivalent digital signal processor. The MCU can also comprise a memory that can further comprise one or more program code segments, as obvious to those skilled in the art, for instructing the MCU to carry the processes as described herein and in more detail below.

In one embodiment, the MCU can be operatively connected to a universal serial bus (“USB”) converter semiconductor chip such as the FTDI Inc. FT232R that can provide wired connectivity to a personal computer. The MCU can also be operatively connected to a wireless receiver semiconductor chip such as the CSR Inc. BlueCore4 for Bluetooth or the Texas Instruments CC2420 for ZigBEE, or any other suitable radio frequency (“RF”) integrated circuit that can also comprise a balun and an antenna, where the antenna can be comprised of a chip, a micro strip antenna or any other antenna as well known to those skilled in the art. The MCU can also be operatively connected to an analog to digital converter that can comprise a resolution of at least 10 bits. In another embodiment, the MCU can be operatively connected to a real time clock semiconductor chip such as the ST Microelectronics M41T65. It is obvious to those skilled in the art that some MCUs can comprise any or all of the above-mentioned functionality integrated internally, which can result in no need for the above-mentioned external chips.

In another embodiment, main controller circuit board (11) can comprise connections to storage media card (22) such as, as an example, a micro-SD card that can be used to store device operation parameters, data collected from the sensors and any pressure adjustments made. Circuit board (11) can also comprise push button switches (23), energy storage disconnect switch (24) that can physically disconnect energy storage device (17) from the rest of the circuitry which is often required for shipping, flexible protective door position detection switch (29), indicator light emitting diodes (LED) (25, 26), USB connectors (27) and wiring harness connector (28).

In one embodiment, pump (12) and valve (13) can be connected to main controller circuit board (11) via wiring harness (21) that can provide solder points to connect the leads from the motor of pump (12) and the valve solenoid of valve (13). Wiring harness (21) can comprise a custom shape to enable the compact design of the apparatus. Wiring harness (21) can connect through connector (28) to main controller circuit board (11).

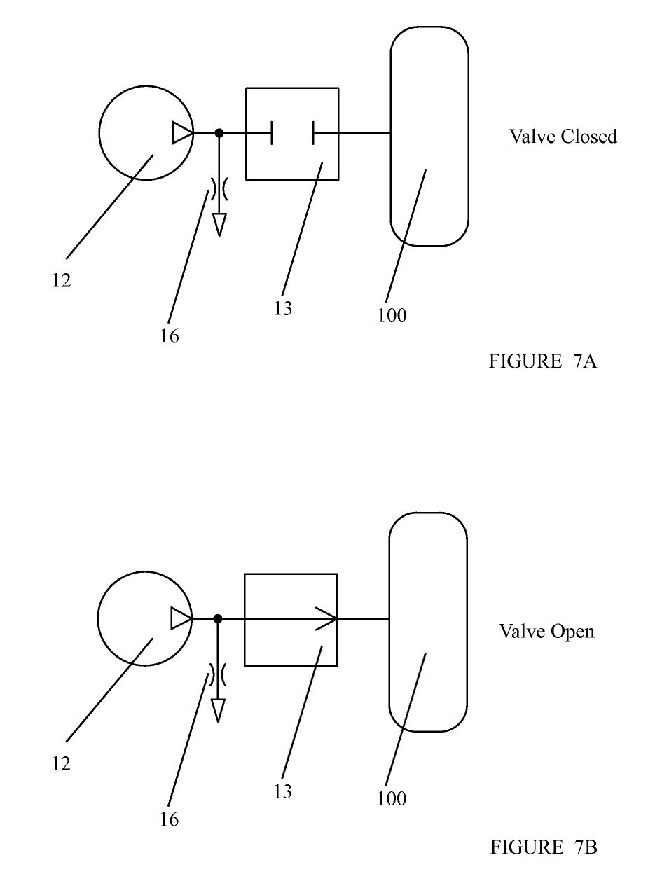

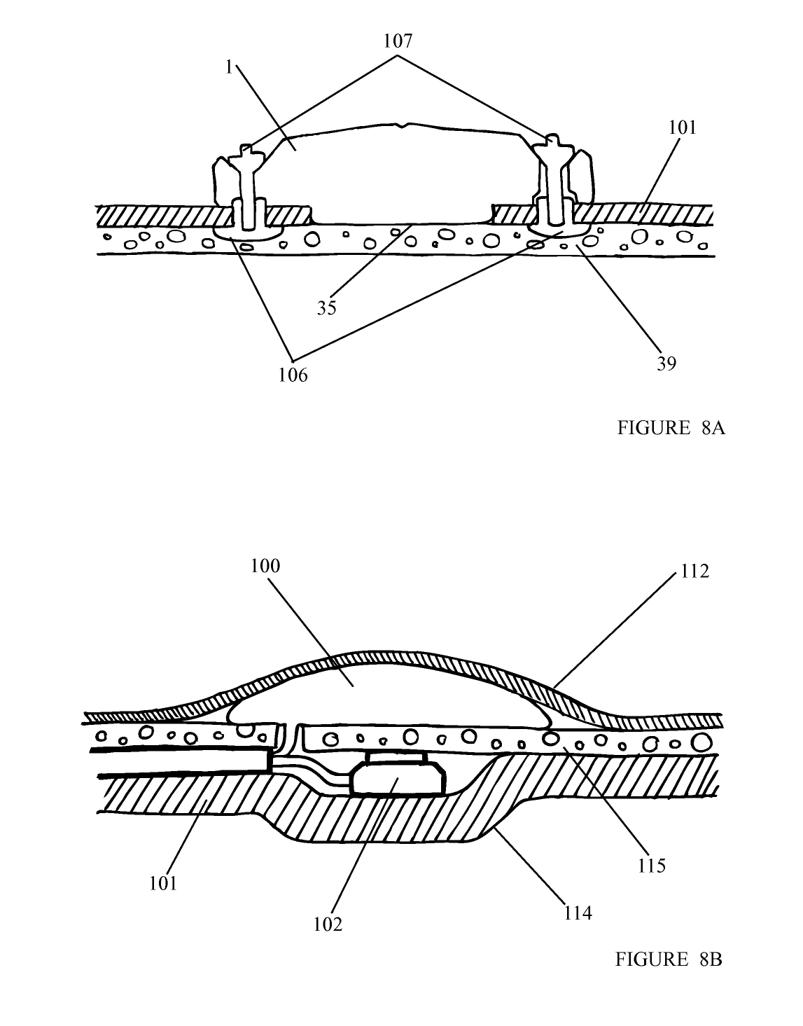

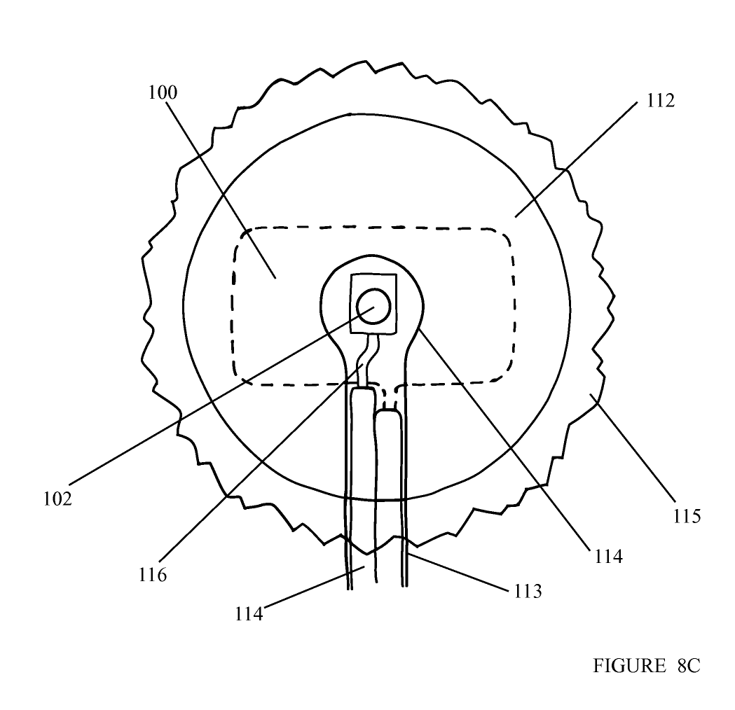

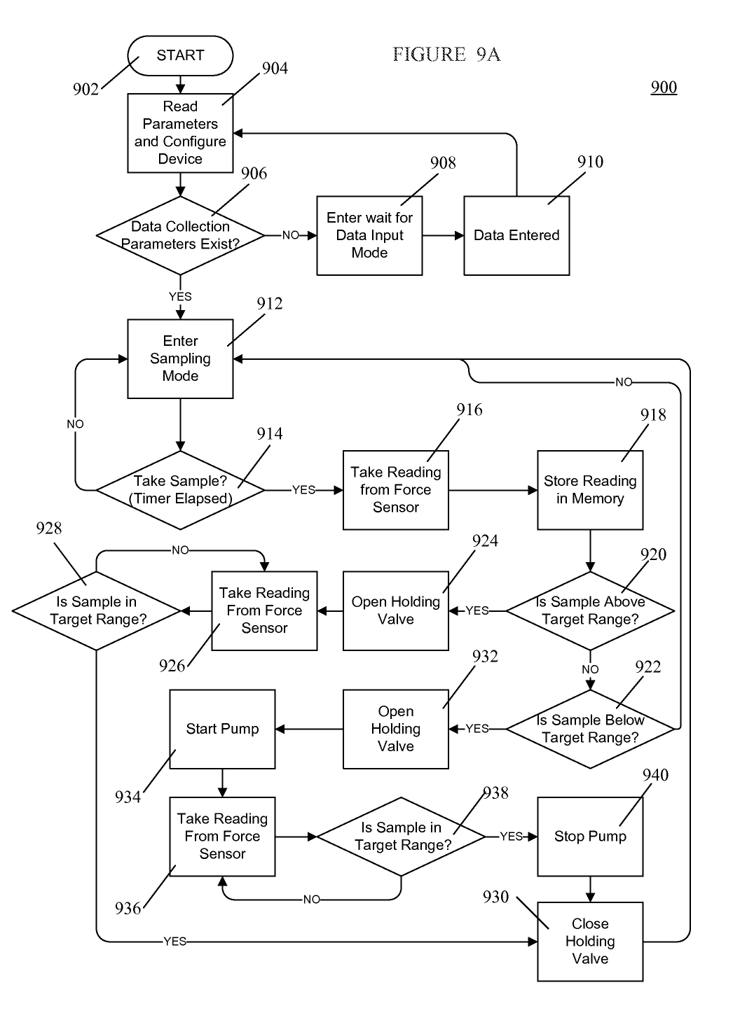

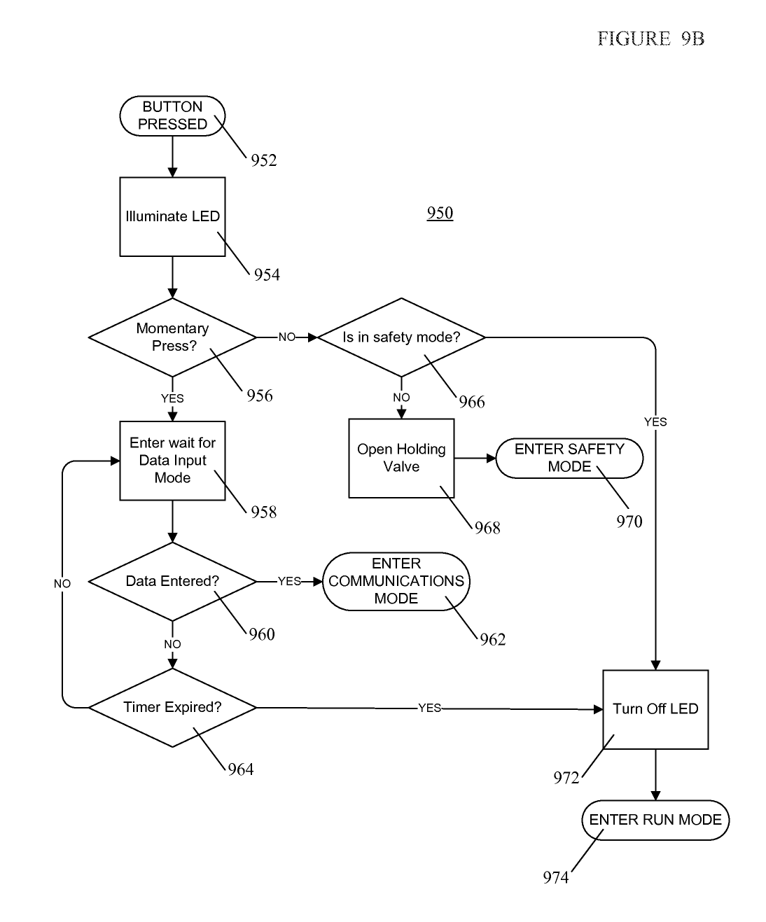

In one embodiment, the air subsystem disposed in main body (1) can increase or decrease the air pressure in air bladder (100). The air subsystem can comprise pump (12), holding valve (13), bleeder valve (16) and interconnect tubing (15). In a representative embodiment, pump (12) can be very small, compact and quiet whilst still providing enough throughput to ensure that air bladder (100) can be inflated. An example of a suitable pump that meets these needs is the Sensidyne Inc., model no. 3A060. In another embodiment, foam pads (38) (open or closed celled-foam) can be placed between the top of plastic enclosure (18A) and the top of pump (12), and between bottom half of enclosure (18B) and pump (12) in order to help dampen the vibration of the motor of pump (12) and, hence, its noise profile. In one embodiment, holding valve (13) can hold the air pressure in air bladder (100) indefinitely whilst being compact. One holding valve that can meet these needs is the Hargraves Inc., model no. 30M05U6.A035V. Interconnect tubing (15) can be made of a material that is both flexible and durable while sealing to the individual air inlets and outlets of the air subsystem. The pneumatic schematics for the air subsystem comprising single pump (12) and single holding valve (13) are shown in FIGS. 7A and 7B.

In other embodiments, the apparatus can use another gas or a fluid instead of air whereby the apparatus can comprise a reservoir for holding gas or fluid to inflate bladder (100), and can further use the same reservoir or a second reservoir for receiving gas or fluid from bladder (100) when it is deflated.

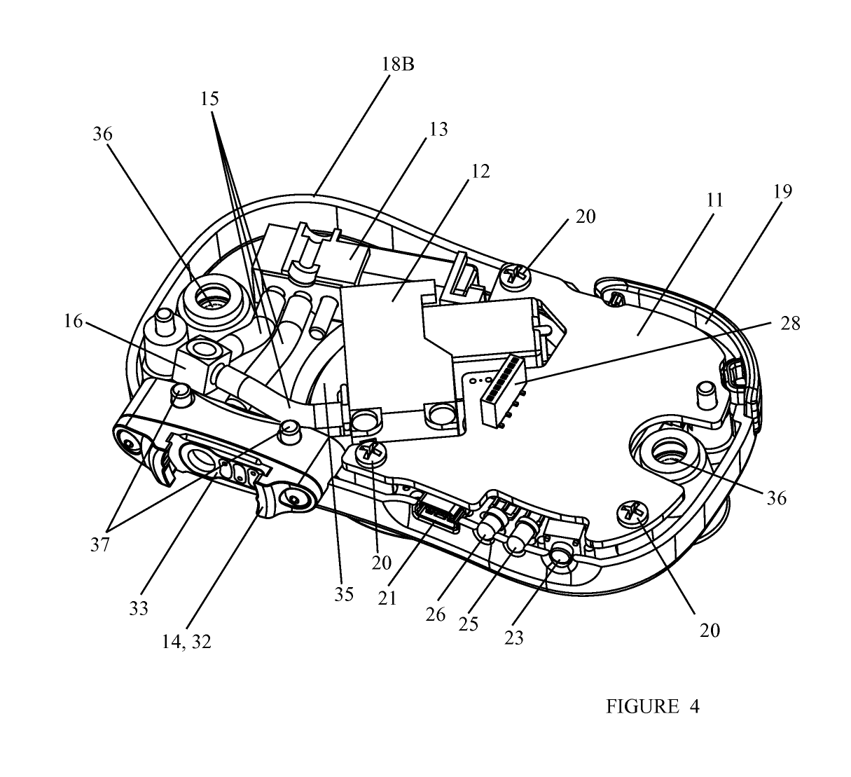

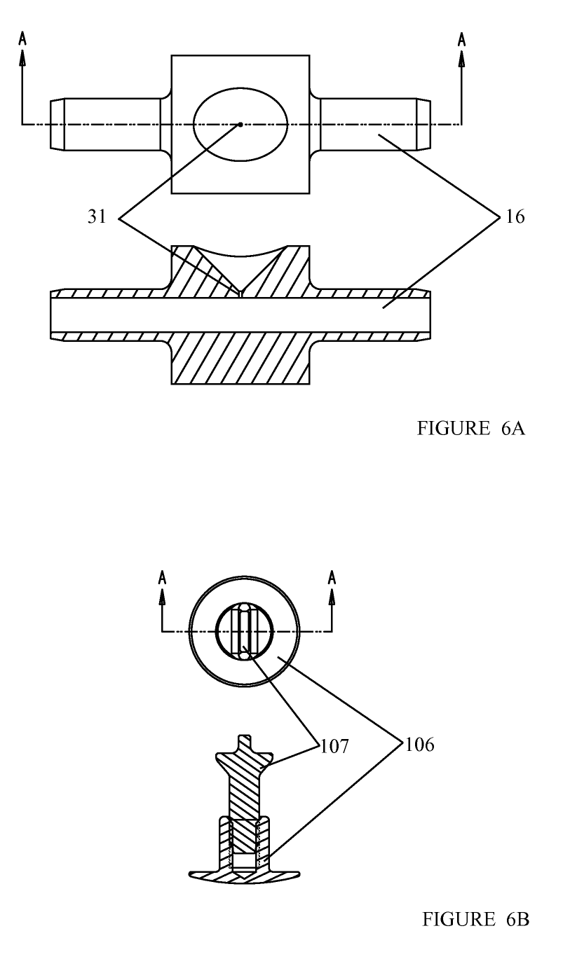

In order to ensure the safety of the patient and to provide for a way for air pressure to release out of the pneumatic air pressure system, the apparatus can comprise an innovative yet inexpensive device that can allow excess air to exhaust out of the system in a safe way. The device can comprise a standard air fitting that can be a straight fitting as shown in FIG. 6A, right angled fitting as shown in FIG. 4, or any other shape or arrangement as may be required, and placing a very small hole (31) into the side of the fitting through to the interior air compartment of the fitting. The fitting comprising small hole (31) acts as a relief or bleeder valve (16) that can ensure that the total pressure of the system never exceeds the designed safe pressure, and can also provides a mechanism for the system to bleed away air to reach the target level. When pump (12) is pumping air to fill bladder (100), air can escape through small hole (31), however, the majority of the air bypasses hole (31) and enters air bladder (100) through the tube. To prevent air from escaping, bleeder valve (16) can be placed after pump (12) but before holding valve (13) such that when holding valve (13) is closed, the air in the air bladder (100) can maintain the correct pressure to ensure target level pressure between orthosis (101) and body (117) of the patient. When holding valve (13) is open, air can escape out of the air bladder (100) through the relief valve (16). Should the patient experience discomfort during the wearing of orthosis (101), the patient can initiate a pressure release event by pressing and holding press button switch (23) on main body (1). The system can wait for a predefined period of time, which can be more than three (3) seconds, and then the system can proceed to open holding valve (13) and allow all of the air to escape. The reason for requiring button switch (23) to be pressed for a period of time before activating is to prevent accidental shut down of the system through an inadvertent depression of the button due to accidental contact or a physical shock event. After the air has all escaped from air bladder (100), which is determined by a period of constant sensor pressure as measured by force sensor (102), holding valve (13) can close and the system can deactivate the air bladder (100) control system but will continue to collect and record force sensor (102) readings. The user can reactivate the air bladder (100) control system by pressing and holding press button switch (23) again for a period of time. When air bladder (100) control system is disabled, both LEDs (25, 26) can be illuminated. After the air bladder (100) control system is reactivated, LEDs (25, 26) can turn off. Other LED (25, 26) messaging sequences are possible. This sequence is depicted in FIG. 9B.

In another embodiment, pressing button switch (23) can move the apparatus out of sampling and correction mode into a mode that allows for interaction with a host main controller computer (referred to as communication mode). If the apparatus is configured to allow for wireless connectivity, a momentary press of the press button switch (23) can turn on the wireless functionality and can cause the apparatus to wait for a predetermined length of time for requests from the computer. If no request is received, the apparatus can re-enter the sampling and correction mode. If the apparatus is in communication mode and press button switch (23) is momentarily pressed again, then the apparatus can enter the sampling and correction mode. In another embodiment of the apparatus, a momentary press of press button switch (23) can create a request to set a new target level for the air pressure in air bladder (100). In a further embodiment of the apparatus, the apparatus can illuminate an LED (25, 26) to indicate to the user that the apparatus is out of regular sampling mode when the apparatus is in any mode other than the sampling and correction mode.

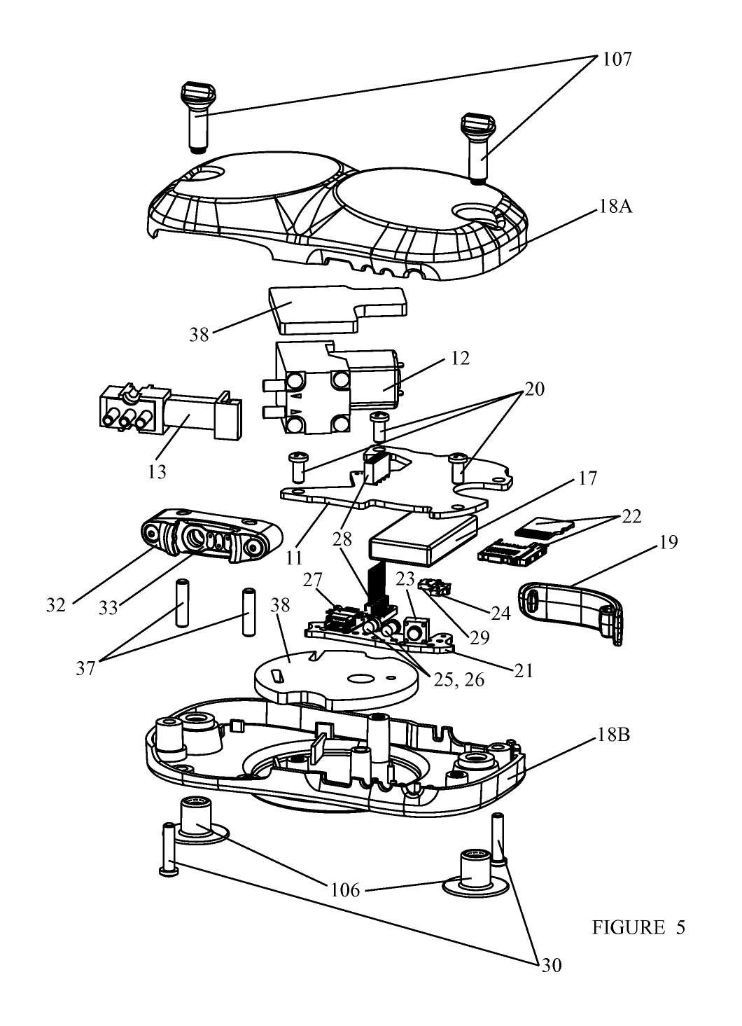

In one embodiment, the main body enclosure can be made up of two halves (18A and 188) that can have a series of openings for access to connectors, switches, LEDs (25, 26), mounting hardware, threaded barbed inserts for assembly of the two halves together and two openings to accommodate flexible protective door (19). Halves (18A) and (18B) can be plastic injection molded parts made of a non-hygroscopic bio-compatible thermoplastic that does not off-gas, that will not promote any kind of mold or bacteria growth, and that has a very good flammability rating. Referring to FIGS. 5 and 8A, the exposed bottom half of plastic enclosure half (18B) can comprise recessed area (34) that is suitable for holding a label indicating regulatory approval status and marketing information. Protruding circular surface (35) is intended to match with a circular hole in orthosis (101) that will help align main body (1) with the mounting holes on orthosis (101). In addition, this protruding circular surface can allow for pump (12) and holding valve (13) to be recessed on the interior to therefore lower the total height of main body (1) that is above the shell of orthosis (101). The bottom half of enclosure (18B) can comprise four holes that go through the surface of the apparatus. Two of the holes are for screws (30) to hold the two enclosure halves together and the second set of holes (36) are to accommodate t-nut (106) which can be used to affix the apparatus to orthosis (101). The interior of the bottom half of the enclosure (18B) can comprise threaded barbed inserts and pins to both align and hold interior components in place. The exterior of the top half of the enclosure (18A) can comprise a stylized surface that is esthetically pleasing. At each end is a hole for t-nut (106) and screw (107) to affix main body (1) to orthosis (101). The interior of the top half of the enclosure (18A) can comprise two threaded barbed inserts which allow the two halves to be connected together with screws (30) and the rest of the interior is generally smooth with a few ribs to add structural support and support interior components such as pump (12) and holding valve (13).

The last component of the exterior of main body (1) is flexible protective door (19) that can prevent dust penetration and can provide access to media card (22). Flexible protective door (19) can have a catch on one side that can be wedged between the two enclosure halves that can act as the hinge for flexible protective door (19), the other side is of flexible protective door (19) can comprise a protruding rectangular section that fits through an opening in enclosure (18a,18B). The protruding section can be slightly oversized at the end to keep flexible protective door (19) from dislodging. This protruding section can also perform a function to engage flexible protective door position detection switch (29). While this flexible protective door position detection switch (29) is engaged (closed), the MCU can store data to media card (22), however if the flexible protective door position detection switch (29) becomes disengaged, then the MCU prepares the media card (22) for removal. Inside of the flexible protective door can be a pin hole which can be used to access and move energy storage disconnect switch (24) to connect energy storage device (17) to the electronic circuitry on the apparatus' first use. Flexible protective door (19) can be an injection molded component that can be made from a flexible non-hygroscopic bio-compatible thermoplastic that does not off-gas, will not promote any kind of mold or bacteria growth, and has a very good flammability rating. The exterior of the apparatus can be cleaned by using a damp cloth or isopropyl alcohol.

In one embodiment, the apparatus can comprise energy storage device (17) such as, but not limited to, a lithium polymer battery that can power all of the systems in the apparatus. Energy storage device (17) must be recharged periodically to ensure consistent and proper operation of the apparatus. Recharging can occur through a variety of different technologies such as but not limited to connecting a wall transformer to a power connector disposed on circuit board (11), connecting a wall transformer to USB port (27), connecting the USB port (27) to a computer USB port capable of supplying power, or some kind of inductive charging requiring no physical contact between the apparatus and the charging station. Although the charging of the apparatus can be done both while the patient is wearing orthosis (101) or not, it would be preferable for the safety of the apparatus and the patient to charge the device while orthosis (101) is not being worn. The apparatus can comprise light emitting diode (LED) (25, 26) that can illuminate during charging, and turn off when charging is complete. Other charging, and charge complete LED (25, 26) signaling can be possible such as having LED (25, 26) come on after charging is complete, or having LED (25, 26) come on when charging and then flash when charging is complete. Charging of energy storage device (17) can be done through a connection to main controller circuit board (11) which can comprise charging circuitry geared towards the type of energy storage device (17) used in the apparatus. For a lithium polymer battery, a semiconductor chip such as the 8Q24010 from Texas Instruments can be used to ensure safe and reliable charging of the battery. As energy storage device (17) runs down, the apparatus can comprise a low battery indicator that can be an LED (25, 26) that can alert the user of the low power condition. In another embodiment, the apparatus can comprise a system to capture the kinetic energy generated by the patient's movements and store that energy to power the remainder of the apparatus.

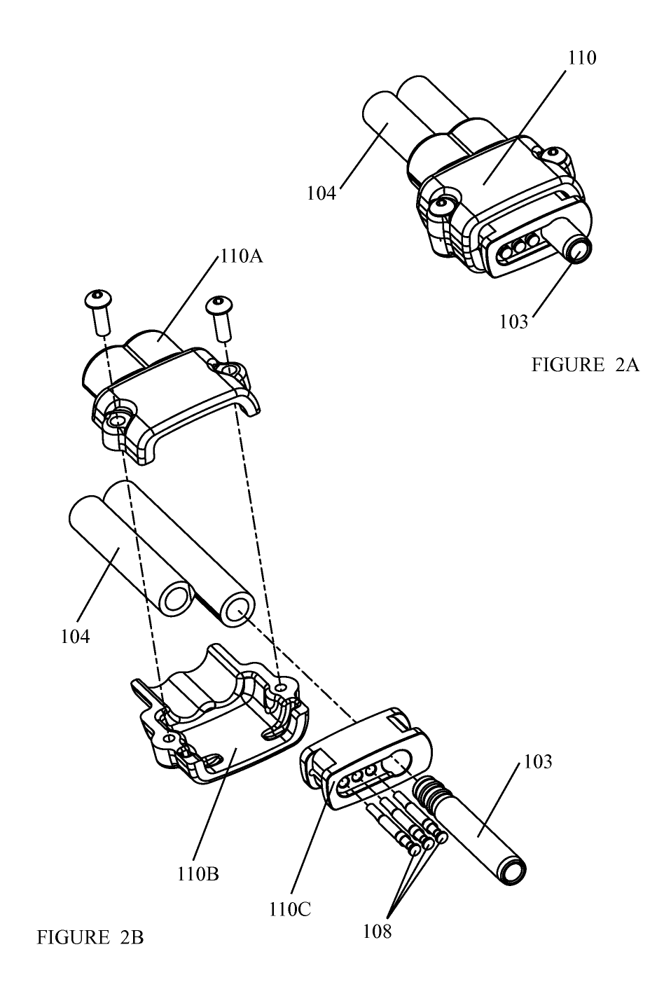

Referring to FIGS. 2A and 2B, one embodiment of sensor/actuator sub assembly (2) can comprise connector (110) for air and electrical signals, force sensor (102), conductors (116), dual channel hose (104) (plastic tubing), and air bladder (100). Connector (110) can comprise of upper half (110A), lower half (110B) and connector insert (110C).

In one embodiment, main body (1) can be connected to force sensor (102) and air bladder (100) with connector (110) and dual channel hose (104) assembly without the need for two separate lines to be run inside of orthosis (101) between the outside shell and the pad. Connector (110) can allow both air and data to be sent through a single dual channeled tube assembly. Dual channel hose (104) can allow for one channel to be used to transfer air from device main body (1) to air bladder (100) with the other hose containing one or more independent conductors, or a cable comprising one or more conductors disposed therein (116) (as shown in FIG. 1C), or multiple cables comprising multiple conductors that can be used to connect force sensor (102) to device main body (1). Device main body (1) side of connector (32) can comprise several components. The first component can comprise a plastic housing which can be aligned with plastic enclosure (18A,18B) of device main body (1) through two dowel pins (37). Plastic enclosure (18A,18B) can comprise several holes protruding through it with one hole containing metal tube (103) (this tube could be made out of any material) which can be used to pass air from device main body (1) internal air delivery system to the dual channel hose (104) and air bladder (100) assembly. The remaining holes can be designed to comprise spring pin connectors (108) also called pogo pins which can be used to make the electrical connection to an interface printed circuit board which can be located on the exterior side of device main body (1) side of connector (32). The plastic housing can also comprise alignment slots at both ends that can mate with the hose side of the connector (110) to hold the connector (110) in place.

Dual channel hose (104) side of connector (110) can comprise plastic housing insert (110C) with a strain relief for the tubing and two small plastic tabs. Plastic housing insert (110C) can comprise a hole inside of it which mates with metal tube (103) on the device main body (1) side of the connector (32). Metal tube (103) can fit snuggly inside of the hole and can further comprise an o-ring seal that can prevent air from escaping. The o-ring can be held in place by a groove. Insert (110C) can also comprise several other holes further comprising spring pin connectors (108) that can make contact with printed circuit board (33) on device main body (1) part of connector (32). To connect, a user can put dual channel hose (104) assembly side of connector (110) perpendicular to device main body (1) side of connector (32) and insert metal tube (103) into the hole and press down until the plastic parts of connector (32, 110) touch. Metal tube (103) can be used as a guide pin to align the two halves of connector (109). Then dual channel hose (104) assembly side of the connector can be rotated around the pivot point created by metal tube (103) until spring pins (108) engage and the tabs on dual channel hose (104) assembly side of connector (110) are held in place underneath of the alignment slots on device main body (1) side of connector (32). To remove connector (110), the user can pivot the dual channel hose (104) assembly side of connector (110) back to perpendicular to device main body (1) side of connector (32) and then pull straight back to disengage metal tube (103).

In one embodiment, air bladder (100) can comprise a disposable neonatal blood pressure cuff (size #4, Critikon Inc.) installed at pressure pad area (111) and secured with neoprene cover (112), as shown in FIG. 8G. Force sensor (102) can be placed underneath air bladder (100) between air bladder (100) and the outside wall of orthosis (101), as shown in FIG. 8B. Referring to FIGS. 1D, 1E and 8A, main body (1) can be installed on orthosis (101) during the manufacturing process when channel (113) can be molded for dual channel hose (104) assembly that terminates on one end at pressure pad area (111) in a bulge (114) that sensor (102) can fit in, and at the other end near the area where main body (1) will be placed. A template can be used to mark the center of the drilled holes where two t-nut (106) mounting nuts will be placed, and the center of the hole-saw cut recessed area where main body (1) can be placed. After the holes are completed, main body (1) can be affixed to orthosis (101) with t-nuts (106) and screws (107). In another embodiment, t-nuts (106) can be covered with foam pad (39). Dual channel hose (104) can be routed to the pressure pad area and force sensor (102) can be placed in recessed area (114) (a protruding bulge when looked at from the outside of orthosis (101)). In a further embodiment, foam pad (115) can be placed on top of force sensor (102) followed by air bladder (100). A neoprene cover can also be placed to cover the whole sensor assembly. With air bladder (100), force sensor (102) and dual channel hose (104) in place, sensor/actuator assembly (2) can be connected to device main body (1).

In another embodiment, the protruding bulge on orthosis (101) that force sensor (102) is located in can be configured such that it can be removed to allow access to force sensor (102) from the back. This could be of benefit in the replacement of a defective force sensor (102) or to remove the sensor when X-Ray radiographs need to be taken of the patient while wearing orthosis (101).

In one embodiment, force sensor (102) can be positioned such that it is mounted against a hard surface on one side of orthosis (101) and air bladder (100) on the other. In other embodiments, a foam pad can be placed between force sensor (102) and air bladder (100). Force sensor (102) can comprise a low profile, a wide force range and a low power consumption rating. Force sensor (100) can be a strain gauge, a load cell or any other technology capable of measuring force known to those skilled in the art. In a representative embodiment, force sensor (102) can comprise an FS01 sensor from Honeywell with a thickness of approximately 8 mm. The FS01 can be sensitive to the forces normal to the orthotic surface, but not to shear forces. The operating force range of the FS01 sensor were measured to be 0-6.82N. Its linearity and hysteresis were ±1.0% Full Scale Span (“FSS”) and ±0.5% FSS, respectively. The full scale span is the algebraic difference between the output voltage at full scale load and the output at zero load. The FSS is dependent upon the supply voltage. The nominal FSS of the FS01 sensor is 3V. The temperature effect on span is ±1.0% FSS at temperature range from 0 to 50° C. The accuracy of the sensor is ±2% FSS (±0.15N). The foam that covers the force sensor (102) can distribute the applied force evenly.

In another embodiment, a pressure sensor located in main body (1) assembly can be used instead of force sensor (102) being used underneath air bladder (100). This would require only air to be transmitted to the pressure pad area. This pressure sensor could be either inline (with an inlet and an outlet), or just connect to the air system with a single inlet. Alternatively, the pressure sensor could be located in the same location as force sensor (102).

In another embodiment, multiple sensor/actuator assemblies (2) could be located throughout orthosis (101). In the case of a scoliosis orthosis, three sensor/actuator assembly (2) can be disposed in orthosis (101) to allow for a three point correction by applying pressure on all three points of the curve (apex and two endpoints). In cases where two sensor/actuator assemblies (2) are used, a curve correction and a de-rotation treatment plan can be implemented.

At an initial fitting of orthosis (101) installed with the apparatus described herein, the patient dons the orthosis (101) and orthosis (101) is tightened to a prescribed tightness. The patient is asked to remember what the tightness “feels” like so that they can tighten orthosis (101) to that force during normal use. After orthosis (101) is fitted to the prescribed tightness, the target pressure level can be stored on the apparatus. Internally, this target pressure level is taken by sampling and then storing the current voltage output of force sensor (102) in the target pressure level field in one or both of the memory in the MCU and media storage card (22). In systems where there are multiple air bladders (100) and multiple force sensors (102), a target force (orthosis-body pressure) level can be stored for each force sensor (102).

[In another embodiment, two or more target levels can be set inside of orthosis (101) when the patient is at different positions such as, but not limited to, standing, sitting or lying down. For each of these positions, the target levels for each force sensor (102) can be stored in the system so that as the patient changes positions, the appropriate target level is used. In these embodiments, the position of the patient can be determined by using a two- or three-dimensional accelerometer disposed on the apparatus. In this case, each target level can have stored a corresponding accelerometer reading on each axis that can be recorded and associated with the target level for each position. Then when the apparatus is checking force samples against the stored target levels to see if a correction is required, the apparatus can also check to see what the current position of the patient is and use the target value corresponding to that position.

As the device is worn by the patient, several pieces of information can be collected for use by the patient's physician and orthotist in analyzing both the quality and quantity of how orthosis (101) is being worn. Quality of orthosis (101) wear is defined as wearing the orthosis (101) at the prescribed tightness, and quantity is defined as the amount of time the orthosis (101) is worn. Both are important in that if orthosis (101) is worn extensively but at too low a tightness, orthosis (101) will likely not meet its treatment goals. The same can be said if the orthosis (101) is worn at the prescribed tightness but not frequently enough to be effective. The two parameters of quality and quantity can be found by analyzing regularly sampled force sensor (102) data from the apparatus. The apparatus can be configured to sample and store force sensor (102) data at virtually any interval that may be desired (including turning off sampling). Caution should be taken to not have the sample interval too frequent as it may cause excessive power consumption, insufficient memory errors and slow data analysis. In addition to force sensor (102) readings, the apparatus can also be configured to record all of air bladder (100) pressure changes that were required during the wearing of orthosis (101). This includes actions where the system increased air bladder (100) pressure as well as those where the system had to decrease air bladder (100) pressure.

The timing of force sensor (102) readings or samples can be set either using an internal timer in the MCU or setting an alarm on the real time clock. The advantage to setting the time on the real time clock is that the MCU can shut down to a very low power mode and then wake up on the alarm interrupt from real time clock. While in the very low power state, the MCU can consume very little power. The MCU can be woken up through a real time clock interrupt, a button press or when the media card (22) door is opened.

The apparatus can operate by recording measurements from force sensor (102) and then comparing those measurements against the target force level to determine if the force reading is within the target force level range.

Referring to FIG. 9A, a flowchart of the Run Mode 900 of the software instructions carried out by the MCU disposed on circuit board (11) is shown. After startup, step 902, the operating parameters can be read and the apparatus can be configured at step 904. If no data collection parameters are found to exist at step 906, the MCU can wait for data to be inputted at step 908 and can receive inputted data at step 910. If data collection parameters are found at step 906, the MCU can enter into sampling mode at step 912. At step 914, a query can be made if a sample has been taken. If “no”, the process can return to step 912. If a sample has been taken, the reading from force sensor (102) can be taken at step 916 and stored in memory at step 918. At step 920, a query can be made if the force reading is above the target range. If “no”, a query can be made at step 922 if the force reading is below the target range. If “no”, the process can return to step 912.

If the force reading is above the target range, the MCU can open holding valve (13) at step 924 and can then take another force reading at step 926. A query can be made at step 928 if the force reading is within the target range. If “no”, the process can cycle between steps 926 and 928 until the air pressure in the system decreases until the force reading is within the target range. Holding valve (13) can then be closed at step 930 and the process can return to step 912.

If the force reading is below the target range, the MCU can open holding valve (13) at step 932 and pump (12) can be started at step 934 to inflate air bladder (100). Force readings can be taken at step 936 and a query can be made at step 938 to determine if the force reading is within the target range. If “no”, the process can cycle between steps 936 and 938 until the air pressure in the system increases to within the target range. Pump (12) can then be stopped at step 940 and holding valve (13) can be closed at step 930. The process can then return to step 912.

This operation of Run Mode 900 is the same for all air bladders (100) in a multiple air bladder (100) system. It is possible to configure holding valve (13) such that it can be either normally opened, meaning that power must be applied to hold the valve closed, or normally closed, meaning that power must be applied to hold the valve open. In order to save power, it can be advantageous to use a normally closed valve as this results in power only being applied to the valve while it is open, which can only occurs briefly during a correction.

In one embodiment, when the average pad force is less than the target level over a period of 15 minutes, pump (12) and holding valve (13) can turn on for 1 second to inflate air bladder (100) after which time holding valve (13) closes. The apparatus can then enter into a fast checking mode sampling rate at once per second. During this mode, fifteen samples can be taken within 30 s and the average force sensor reading can be compared to the acceptable range (which is set by the patient's orthotist). If the average reading is within the acceptable range, the apparatus can return to the normal sample mode with a predetermined sample rate, as selected by the clinician. In one embodiment, the sampling rate can be once per minute. Otherwise, pump (12) and holding valve (13) can be activated again and more air can be pumped into air bladder (100) by pump (12). This procedure can be repeated until the acceptable target force level is attained. However, if the pressure is beyond the acceptable range, holding valve (13) can open for 0.5 s to release air from air bladder (100). The apparatus can then enter a fast checking mode again with the sampling rate at once per second. This procedure can be repeated until the acceptable target force level is attained.

In another embodiment, when orthosis (101) is not being worn, to prevent it from continuously pumping air into air bladder (100) indefinitely and thus running down energy storage device (17) such as, but not limited to a battery, the apparatus can interpret force sensor (102) samples far below the target force level to mean that orthosis (101) is either too loose for air bladder (100) to compensate or that the patient does not have orthosis (101) on. In this situation, the apparatus can enter into a Safety Mode, and record the sample and execute no action. The apparatus can then continue to sample force sensor (102) at its regular sampling interval and only when the patient puts orthosis (101) back on will the force (body-orthosis pressure) correction system be reactivated. In one embodiment, the apparatus can further comprise a vibratory motor that would initiate if the apparatus has made several attempts to bring the air pressure in air bladder (100) to the target level but has been completely unsuccessful. This vibratory motor could also be used by the apparatus to inform the user of other information about their orthosis (101) wear or of any alarm condition or failure of the operation of the apparatus.

Referring to FIG. 9B, the process steps that can be carried out by the apparatus when button (23) is depressed is shown as process 950. When button (23) is depressed at step 952, LED (25) and/or (26) can illuminate at step 954. At step 956, a query can be made whether the button (23) was momentarily pressed or if it was pressed and held for a predetermined period of time. If button (23) was pressed momentarily, the apparatus can wait for data to be entered at step 958. A query can be made at step 960 if data has been entered. If “yes”, the apparatus enters a Communications Mode at step 962. If “no”, process 950 can cycle through steps 958, 960 and 964 until data is entered, or until a timer of a predetermined period of time has expired upon which process 950 can proceed to step 972 to turn off LED (25,26) and return to Run Mode at step 974.

If button (23) is pressed and held, process 950 can move from step 956 to step 966 where a query can be made whether apparatus is in Safety Mode. If “yes”, process 950 can proceed to steps 972 and 974 to turn off LED (25, 26) and return to Run Mode. If “no”, process 950 can proceed to step 968 to open holding valve (13) and enter the Safety Mode at step 970.

Another scenario that can be monitored by the apparatus is if the patient tightens orthosis (101) far too tightly. In this case, holding valve (13) can open and whatever air is in air bladder (100) will exhaust out through pressure relief valve (16). In order to prevent holding valve (13) from staying open indefinitely if the body-orthosis pressure is still far above the target pressure level, the apparatus can detect when holding valve (13) is open and the pressure remains constant for a certain predefined period of time and can then close holding valve (13) and wait for orthosis (101) pressure to go below the target pressure level.

In another embodiment, the apparatus can comprise a temperature sensor operatively connected to the MCU to detect whether or not the patient has removed orthosis (101). This can be used in combination with or in place of force sensor (102) to determine an accurate measurement of compliance. The temperature sensor can be placed either on the inside wall of main body (1) on the body (117) side of the enclosure (18A,18B), or externally between orthosis (101) padding and the shell of orthosis (101). While the patient is wearing orthosis (101), a constant temperature can be measured. When the patient removes orthosis (101), the temperature will generally fall (or change) and the apparatus can measure this temperature change to determine if the patient has been wearing orthosis (101) is accordance with the regime programmed into the appliance by the patient's physician and/or healthcare professional.

As the patient progresses through their treatment, it may be required that device main body (1) part of the apparatus needs to be removed whilst maintaining air bladder (100) pressure. This is often required for in-orthosis (101) X-Ray images to be taken. To accomplish this, a clip can be used to compress and close the air side of soft dual channel hose (104) between connector (110) and air bladder (100). Often, this can be done by folding the dual channel hose (104) over itself to create a kink and then applying the clip at the center of the bend. After the air side of dual channel hose (104) has been clipped, air/data cable connector (110) can be removed from device main body (1). Now with air bladder (100) and sensor disengaged from main body (1) of the apparatus, main body (1) can be removed from orthosis (101) itself, a task completed by loosening and removing screws (107) from plastic t-nuts (106) that are affixed to the inside of orthosis (101) and then pulling main body (1) away from orthosis (101). To re-attach main body (1) to orthosis (101), the above procedure can be reversed.

In an alternative embodiment, t-nuts (106) can be replaced by two or more tabs that can engage with slots disposed on orthosis (101) that can then be turned a quarter turn thereby locking into orthosis (101). Materials with low X-Ray absorption properties can be used to ensure the X-Ray absorption by the components of the device that remain on orthosis (101) are as little as possible so as not to block X-Ray detail.

In another embodiment, a flapper valve can be connected to dual channel hose (104) side of connector (110) that can act as a back flow valve when connector (110) is disconnected from main body (1). In yet a further embodiment, connector (110) can comprise the flapper valve internally such that the flapper valve is open when connector (110) is connected to main body (1) but then closes when connector (110) is disconnected from main body (1) and just as connector (110) leaves the main body side of connector (32) air tube. The air pressure in dual channel hose (104) and air bladder (100) are higher than atmospheric pressure which forces the flapper against a seal and keeps air from escaping from dual channel hose (104) and air bladder (100). When dual channel hose (104) side of connector (110) is engaged again, the air tube on the main body side of connector (32) can cause the flapper valve to fold up and out of the way allowing air to flow freely between main body (1) and air bladder (100).

The apparatus can also comprise one or more interfaces that can be used to transmit to, and receive data from, a main controller personal computer (not shown). In one embodiment, main body (1) can comprise mini-USB port (27) to connect to the main controller personal computer via a USB cable. Mini-USB port (27) can be located inside of an access door disposed on main body (1). In another embodiment, the apparatus can comprise short range wireless technology such as but not limited to ZigBEE® or Bluetooth®. Wireless communication requires there to be transceiver on the main controller personal computer using the same technology as that used on the apparatus. In a further embodiment, data transfer can be effected by the apparatus placing the data on removable storage media card (22) whereby storage media card (22) can be removed from main body (1) and inserted into the main controller personal computer directly. Examples of suitable storage media cards (22) can be, but not limited to, an SD or micro-SD card as well known to those skilled in the art.

Once the apparatus is connected to the main controller personal computer through one of the aforementioned interfaces, the main controller personal computer can now read the current device operational configuration and the data stored in the apparatus. In the case of the connection with the USB port (27) or Wireless connection, the data can be streamed from the apparatus to the main controller. In the case of media card (22), the data can be read in as a file. The data can also be uploaded to a website or other network endpoint via the main controller personal computer through the Internet. For the serial connections including, but not limited to, USB port (27) and Wireless, a protocol can be implemented developed to provide a query and response structure such that operational configuration parameters can be set and queried data can be read and cleared as required. This protocol can include, but is not limited to, a simple packet based system or a more complex XML messaging scheme. In other embodiments, the apparatus can use power conservation techniques in order to conserve power. These can include the apparatus powering down the wireless and USB components on the MCU before the apparatus can enter in a Communication Mode, as shown in FIG. 9B, by a momentary press of press button (23).

Some of the apparatus parameters that can be configured include, but are not limited to, the current date and time, and on a per force sensor (102) basis: the sampling rate, the number of samples outside of the target range to be taken before a correction is initiated, force target level for all standardized positions, and the size of the target range (target level+/−X %). The device parameters can be stored in, but not limited to, a memory component disposed on the MCU, such as an EEPROM, battery backed up memory either inside the MCU or as part of another chip disposed on circuit board (11), flash memory operatively connected to the MCU, or on media card (22). The collected data that can be transmitted from the apparatus can include time stamped force sensor (102) samples, time stamped body position, and time stamped correction information. This collected data can be stored in, but not limited to, the EEPROM on the MCU, battery backed up memory either inside the MCU or as part of another chip, flash memory connected to the chip, or on media card (22). It is possible for the data collected by the apparatus and the current parameters of the apparatus to be collected and then transmitted to the patient's physician or healthcare giver through a communications network such as, but is not limited to, the Internet. After receiving this information, the patient's physician or healthcare giver can then analyze the wear characteristics, provide information to the patient, and change the treatment protocol if necessary by sending new parameters to the apparatus remotely thus preventing the need for the patient to travel and meet with their physician or healthcare giver.

In another embodiment, the apparatus can comprise a boot loader and firmware update mechanism that can provide for after manufacture reprogramming of the firmware that controls the apparatus while simultaneously providing a failsafe mechanism that can reinstate and recover the apparatus in the event that a programming cycle goes awry. This firmware reprogramming sequence can be triggered through, but is not limited to, either a wireless or a USB interface disposed on the apparatus. In other embodiments, this reprogramming process can be done through software operatively installed and running on the connected main controller personal computer.

In another embodiment of the apparatus, the patient can use the apparatus' wireless connection to connect to a secondary device, such as but not limited to, a mobile phone, a desktop computer, a touchpad computer, a netbook computer, a laptop computer, a television set top box, a vehicle, or a watch, to view the current status of the patient's treatment and the data collected while the patient has been wearing the orthosis (101). This secondary device can also comprise information and means to display advice on how patient can wear orthosis (101) in accordance with the protocol and regimen the patient is to follow in wearing orthosis (101) and to alert the patient as to information concerning the patient's habits in wearing orthosis (101) and the apparatus itself. This information can include, but is not limited to, the amount of time orthosis (101) is worn, the current power status of the battery disposed on the apparatus and the amount and type of data collected. In a further embodiment, the secondary device can comprise a training tool to help the patient ensure that orthosis (101) is being worn at the prescribed target level tightness. All of this information can be presented on the secondary device through a graphical or textual display, verbal notification, or any other information dispersal technology obvious to those skilled in the art.

In other embodiments, the apparatus described herein can further comprise a set of instructions for installing the apparatus on an orthosis, and for configuring and operating the apparatus. In these embodiments, the apparatus can be provided as a kit for installation on a new orthosis during its manufacture, or for retro-fitting an existing orthosis.

While the apparatus and method described has been developed and applied in the treatment of scoliosis, it is obvious to those skilled in the art that apparatuses, methods, devices and techniques described herein can be extended and applied to any orthotic brace used to correct other physiological ailments.

Although a few embodiments have been shown and described, it will be appreciated by those skilled in the art that various changes and modifications might be made without departing from the scope of the invention. The terms and expressions used in the preceding specification have been used herein as terms of description and not of limitation, and there is no intention in the use of such terms and expressions of excluding equivalents of the features shown and described or portions thereof, it being recognized that the scope of the invention is defined and limited only by the claims that follow.

### REFERENCES

This application incorporates the following documents [1] to [12] by reference in their entirety.
[1] C. J. Goldberg, D. P. Moore D P, E. E. Fogarty, E. E. Dowling, The effect of brace treatment on the incidence of surgery, Spine 26 (2001), 42-47.
[2] Karol La. Effectiveness of bracing in male patients with idiopathic Scoliosis, Spine 2001; 26:2001-2005.
[3] Fernandez-Feliberti R; Flynn J; Ramirez N; Trautmann M; Alegria M. Effectiveness of TLSO bracing in the conservative treatment of idiopathic scoliosis. J Pediatr Orthop. 1995 March-April; 15(2):176-81.
[4] Lonstein J E and Winter R B. The Milwaukee brace for the treatment of adolescent idiopathic scoliosis. A review of one thousand and twenty patients. J Bone Joint Surg Am 76, 1207-1221, 1994.
[5] Rowe D E, Bernstein S M, Riddick M F, Adler F, Emans J B and Gardner-Bonneau D. A meta-analysis of the efficacy of non-operative treatments for idiopathic scoliosis. J Bone Joint Surg Am 79, 664-674, 1997.
[6] Wong M S, Mak A F, Luk K D, Evans J H, Brown B. Effectiveness and biomechanics of spinal orthoses in the treatment of adolescent idiopathic scoliosis (AIS). Prosthetics & Orthotics International 2000; 24:148-162.
[7] Havey R., Gavin T, Patwardhan A, Pawelczak S., Ibrahim K., Andersson G., Lavender S. A Reliable and Accurate Method for Measuring Orthosis Wearing Time, Spine 2002; 27:211-4.
[8] Lou E, Raso V J, Hill D L, Durdle N G, Mahood J K, Moreau MJ. The Daily Force Pattern of Spinal Orthoses in Subject with Adolescent Idiopathic Scoliosis. Prosthetics & Orthotics International, 2002; 26:58-63.
[9] Lou E, Raso V J, Hill D L, Durdle N G, Mahood J K, Moreau M J: Correlation between quantity and quality of Orthosis wear and treatment outcomes in AIS. Prosthetics and Orthotics International Journal, 2004; 28:49-54.
[10] Jiang H, Raso V J, Hill D L, Durdle N G, Moreau M. Interface Pressures in the Boston Brace Treatment for Scoliosis. Proceedings Int'l Symposium on 3-D Scoliotic Deformities, Montreal 1992 Jun. 27-30: 395-399.
[1]) Lavelle J R, Smith K, Platts R, Morley T R, Ransford A O, Edgar M. An Assessment of Brace Compliance in Adolescent Idiopathic Scoliosis using a New Brace Timer. J. Bone Joint. Surg. 1996; 78B:162.
[12] Nicholson G, Ferguson-Pell M., Smith K., Edgar M., Morley T. Development of Instrumented spinal Brace for measuring compliance and skin microclimate in the conservative treatment of adolescent idiopathic scoliosis. Proceeding of British Scoliosis Society, Silver Jubilee Meeting, 2001, March 7-9, p. 17.

We claim:

1. An apparatus for monitoring and controlling the pressure applied to a patient by an orthosis, comprising:
a) a main body assembly configured to be operatively disposed on the orthosis, the main body assembly further comprising:
i) an enclosure configured to be releasably attached to the orthosis,
ii) an air pump disposed within the enclosure,
iii) an air holding valve disposed within the enclosure and operatively connected to the air pump,
iv) a first connector disposed on the enclosure and operatively connected to the air holding valve, the first connector further comprising at least one electrical connector,
v) a controller disposed within the enclosure for controlling the air pump and the air holding valve, the controller operatively connected to the at least one electrical connector, and
vi) an energy source device disposed within the enclosure and operatively connected to the controller, the air pump and the air holding valve; and
b) a sensor/actuator assembly, comprising:
i) at least one air bladder configured to be disposed on the orthosis, the at least one air bladder further configured to provide pressure to the patient,
ii) at least one force sensor operatively disposed on the at least one air bladder, the at least one force sensor configured to measure the pressure the at least one air bladder exerts on the patient wearing the orthosis,
iii) a hose assembly having first and second ends, the first end operatively connected to the at least one air bladder and the at least one force sensor, the hose assembly further comprising an air hose operatively connected to the at least one air bladder and a conduit operatively connected to the at least one force sensor,
iv) a second connector operatively connected to the second end of the hose assembly and configured to releasably connect with the first connector, the second connector configured to provide communication from the air holding valve to the at least one air bladder when the second connector is connected to the first connector, and
v) at least one electrical conductor disposed within the conduit operatively connecting the at least one force sensor to the second connector whereby an electrical connection is made between the at least one force sensor and the controller when the second connector is connected to the first connector.

2. The apparatus as set forth in claim 1, wherein the controller comprises a microcontroller disposed on a circuit board, the microcontroller comprising a central processing unit (“CPU”) and a memory operatively connected to the CPU, the memory comprising at least one program code segment comprising instructions for controlling the microcontroller to control the air pump and the air holding valve, and to receive and store data in the memory.

3. The apparatus as set forth in claim 2, wherein the memory comprises one or more of the group consisting of an electrically erasable programmable read-only memory (“EEPORM”) disposed on the microcontroller, a memory chip disposed on the circuit board and operatively connected to the microcontroller, and a removable media storage card inserted in a media card connector disposed on the circuit board, the media card connector operatively connected to the microcontroller.

4. The apparatus as set forth in claim 2, further comprising means for inputting operating parameters to the microcontroller whereby the microcontroller can control the air pump and the air holding valve to inflate the at least one air bladder to a predetermined pressure.

5. The apparatus as set forth in claim 2, wherein the microcontroller further comprises means for reading the air pressure of the at least one air bladder at predetermined intervals and storing the air pressure reading in the memory.

6. The apparatus as set forth in claim 5, further comprising means for transmitting the air pressure reading to a secondary device configured to communicate with the microcontroller to receive the air pressure reading, and to transmit configuration parameters and operating parameters to the memory to be used by the microcontroller in operating the apparatus.

7. The apparatus as set forth in claim 6, wherein the secondary device comprises one or more of the group consisting of a mobile phone, a desktop computer, a touchpad computer, a netbook computer, a laptop computer, a television set top box, a vehicle and a watch.

8. The apparatus as set forth in claim 1, further comprising at least one bleeder valve operatively disposed between the air holding valve and the first connector.

9. The apparatus as set forth in claim 1, further comprising fasteners for releasably fastening the main body assembly to the orthosis.

10. The apparatus as set forth in claim 1, further comprising a set of instructions for installing the main body assembly and the sensor/actuator assembly on the orthosis, and for configuring and operating the apparatus.

11. The apparatus as set forth in claim 1, further comprising the orthosis.

12. A method for monitoring and controlling the pressure applied to a patient by an orthosis disposed on the patient, the method comprising the steps of:
a) measuring the air pressure of at least one air bladder disposed on the orthosis for applying pressure to the patient; and
b) inflating the at least one air bladder with air until the air pressure is within the predetermined air pressure range if the air pressure is below a predetermined air pressure range.

13. The method as set forth in claim 12, further comprising the step of deflating the at least one air bladder if the air pressure is above the predetermined air pressure range until the air pressure is within the predetermined air pressure range.

14. The method as set forth in claim 12, further comprising the steps of:
a) determining the physical position of the patient; and
b) inflating or deflating the air pressure in the at least one air bladder until the air pressure is within the predetermined air pressure range corresponding to the physical position of the patient.

15. The method as set forth in claim 12, further comprising the steps of:
a) reading and recording the air pressure of the at least one air bladder at predetermined intervals; and
b) transmitting the air pressure readings to a secondary device for receiving the air pressure readings.

16. The method as set forth in claim 15, wherein the secondary device comprises one or more of the group consisting of a mobile phone, a desktop computer, a touchpad computer, a netbook computer, a laptop computer, a television set top box, a vehicle and a watch.

17. An apparatus for monitoring and controlling the pressure applied to a patient by an orthosis disposed on the patient, comprising:
a) means for measuring the air pressure of at least one air bladder disposed on the orthosis for applying pressure to the patient; and
b) means for inflating the at least one air bladder with air until the air pressure is within the predetermined air pressure range if the air pressure is below a predetermined air pressure range.

18. The apparatus as set forth in claim 17, further comprising means for deflating the at least one air bladder if the air pressure is above the predetermined air pressure range until the air pressure is within the predetermined air pressure range.

19. The apparatus as set forth in claim 17, further comprising:
a) means for determining the physical position of the patient; and
b) means for inflating or deflating the air pressure in the at least one air bladder until the air pressure is within the predetermined air pressure range corresponding to the physical position of the patient.

20. The apparatus as set forth in claim 17, further comprising:
a) means for reading and recording the air pressure of the at least one air bladder at predetermined intervals; and
b) means for transmitting the air pressure readings to a secondary device for receiving the air pressure readings.

21. The apparatus as set forth in claim 20, wherein the secondary device comprises one or more of the group consisting of a mobile phone, a desktop computer, a touchpad computer, a netbook computer, a laptop computer, a television set top box, a vehicle and a watch.

22. The apparatus as set forth in claim 17, further comprising the orthosis.
